(19) United States
(12) Patent Application Publication
(10) Pub. No.: US 2022/0257904 A1
(43) Pub. Date: Aug. 18, 2022

(54) Methods And Devices For Establishing A Connection Between Adjacent Anatomical Spaces Using Magnets

(71) Applicant: NXT Biomedical, LLC, Irvine, CA (US)

(72) Inventors: Joseph Passman, Irvine, CA (US); Alexander Siegel, Irvine, CA (US); Glen Rabito, Irvine, CA (US); Stanton J. Rowe, Irvine, CA (US); Elliot Howard, Irvine, CA (US); Robert C. Taft, Irvine, CA (US); Robert S Schwartz, Irvine, CA (US)

(73) Assignee: NXT Biomedical, LLC, lrvine, CA (US)

(21) Appl. No.: 17/597,830

(22) PCT Filed: Jul. 30, 2020
(86) PCT No.: PCT/US20/44347

§ 371 Date: Jan. 25, 2022

Related U.S. Application Data
(60) Provisional application No. 62/881,239, filed on Jul. 31, 2019.
(60) Provisional application No. 62/906,001, filed on Sep. 25, 2019.

(51) Int. Cl. A61M 25/01; A61B 17/34; A61M 25/10

(52) U.S. Cl. CPC A61M 25/0127; A61B 17/3478; A61B 2017/00876; A61M 25/10; A61M 25/0194

(57) ABSTRACT

Establishing connections across tissue walls used to create shunts or similar passageways are formed using methods and devices that utilize magnets for navigation and location of devices on opposite sides of the walls. Various tools are provided that optimize the uses provided by the magnets.

### RELATED APPLICATIONS

This application claims priority to Provisional Patent Application Ser. No. 62/881,239, filed Jul. 31, 2019, entitled Coronary Artery-LAA Shunt; and, Provisional Patent Application Ser. No. 62/906,001, filed Sep. 25, 2019, entitled LAA Flow Augmentation, all of which are hereby incorporated herein by reference in their entireties.

### BACKGROUND OF THE INVENTION

Internal medical procedures are typically either performed surgically or trans-catheterally (percutaneously), with the latter being relatively new due to the significant innovation that has occurred in this field over past few decades. This innovation has been driven by the significant advantages presented by percutaneous procedures—decreased risk of infection, significantly decreased scarring, significantly decreased patient trauma, reduced amounts and durations of anesthesia, reduced occurrence of complications during and after the procedure, significantly faster recovery times, reduction in the need for post-procedural pain medications, just to name a few.

The disadvantages, however, arise during more complicated procedures and involve the physician's inability to directly see and manually manipulate the targeted area. Additionally, as mentioned above, percutaneous procedures involve highly specialized tools that must be able to navigate to the targeted area and manipulate tissue in order to perform the desired procedure.

With regard to visualization, some procedures, especially those performed on the inside of lumens, are performed using imaging technology such as fluoroscopy. Other procedures that are performed on the outsides of organs or bodily passageways use endoscopes to provide a live visual image. These procedures often involve inflating a body cavity with a gas to provide viewing and illumination space to allow the camera to focus.

Procedures that involve the shunting of one lumen to another lumen are often performed surgically due to the unique challenges presented to performing them percutaneously. A percutaneous approach would necessarily involve a first catheter being navigated through a first lumen and a second catheter being navigated through a second lumen. The two catheters would then need to be brought into close proximity and the shunt would have to be created or installed without allowing the fluids carried in either lumen from being spilled into the bodily cavity between the two lumens.

There is thus a need for a device and method that may be used to perform procedures involving the implantation of shunts between multiple lumens.

### OBJECTS AND SUMMARY OF THE INVENTION

The present invention is directed toward devices and methods that use magnets to assist in navigation and connection or communication between two or more percutaneous devices and/or implants.

In one aspect, magnets may be used for temporary guidance and placement use or may be permanently implanted.

In another aspect, the magnets are circular or have other geometric shapes including, but not limited to, a torus. The magnets are placed in both chambers and lumens that are desired to be placed in apposition.

Another aspect of the invention provides catheters with magnets mounted thereon and used to provide points of shunt contact or placement.

In at least one embodiment, the magnetic attraction will pull tissues and catheters together in a fixed, reliable geometric manner.

In at least one embodiment, a perforating needle, wire, or other tool centered or proximate the magnets can then be reliably made to transfer opposing anatomical structures in ideal locations. The magnet will thus serve as a guidance method using “hole” or other patterns on which to guide the placement of the shunt.

In at least one embodiment, the magnets may be released and removed from the body at the procedure's end. In other embodiments, the magnets may be left in place, for example, if a chronic implant is required. If left in place, the magnets may cover with tissue and become embedded in a tissue covering, or experience ingrowth. Texturing the magnet surface or adding a textured coating or a chemical surface coating may facilitate ingrowth.

One aspect of the invention provides magnets having flat mating surfaces that spread the pressure of the connection in order to prevent adverse tissue shear and tearing, especially compared to typical point connections such as sutures and other conventional connection methods.

One aspect of the invention provides shunt devices that have lips or flanges that contain magnets or may be magnetized. A magnetic shunt may provide attraction forces that hold the tissues together chronically in a desired configuration.

Another aspect of the invention provides a mechanical backbone or structure of the device that prevents physiologic expansion of the shunt, which could result in two halves of the shunt growing at unnatural or adverse diameters.

One aspect of the invention provides a system for establishing a connection across tissue walls of adjacent anatomical spaces comprising a first catheter having a first lumen and carrying a tool in said lumen; a second catheter having a second lumen; a first magnetic feature on a distal end of said first catheter; a second magnetic feature on a distal end of said second catheter; wherein said first magnetic feature includes a polar arrangement configured to attract said second magnetic feature; a first aperture on said first catheter and a second aperture on said second catheter; wherein said first and second apertures align to form a passageway when said first and second magnetic features are connected across said tissue walls; wherein said tool is positioned to puncture said tissue walls when advanced through said first lumen; a tool receiver associated with said second catheter for engaging the tool after it crosses through said tissue walls in order to establish a connection across the tissue walls.

Another aspect of the invention is a method of forming a connection across tissue walls of adjacent anatomical spaces comprising: navigating a first catheter to a first target location in a first anatomical space; navigating a second catheter to a second target location in second anatomical space adjacent said first anatomical space; using magnetic force to create a connection between said first and second catheter across tissue walls of said first and second anatomical spaces; passing a tool through the tissue walls from said first catheter to said second catheter thereby establishing a mechanical connection; removing said first and second catheters.

Yet another aspect of the invention is a system for establishing a connection across tissue walls of adjacent anatomical spaces comprising: a first navigable device having a first magnetic feature on a distal end thereof; a second navigable device having a second magnetic feature on a distal end thereof; wherein said first and second magnetic features are attracted to each other across tissue walls such that said distal ends of said first and second navigable devices become temporarily fixed to each other when brought into magnetic proximity of each other; a tool passable through tissue from one of said first and second navigable devices to the other, thereby establishing a mechanical connection across the tissue walls.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a diagram of a heart including a pair of catheters that generally demonstrate an aspect of the invention;

FIG. 2 is a side cutaway of an embodiment of the invention;

FIG. 3 is an end view of an embodiment of the invention;

FIG. 4 is a side view of an element of the invention;

FIG. 5 is a perspective view of an embodiment of the invention;

FIG. 6 is a side view of an embodiment of the invention;

FIG. 7 is a side view of a step of an embodiment of a method of the invention;

FIG. 8 is a side view of a step of an embodiment of a method of the invention;

FIG. 9 is a side view of a step of an embodiment of a method of the invention;

FIG. 10 is a side view of a step of an embodiment of a method of the invention;

FIG. 11 is a side view of a step of an embodiment of a method of the invention;

FIG. 12 is a perspective view of an embodiment of a device of the invention;

FIG. 13 is a perspective view of a distal end of an embodiment of a device of the invention;

FIG. 14 is a perspective view of a component of an embodiment of the invention;

FIG. 15 is a perspective view of an embodiment of a device of the invention;

FIG. 16 is a perspective view of a component of an embodiment of the invention;

FIG. 17 is a perspective view of a component of an embodiment of the invention;

FIG. 18 is an end view of a component of an embodiment of the invention;

FIG. 19 is an end view of a component of an embodiment of the invention;

FIG. 20 is a perspective view of a component of an embodiment of the invention;

FIG. 21 is cutaway side view of an embodiment of the invention;

FIG. 22 is a perspective view of a component of an embodiment of the invention;

FIG. 23 is a plan view of an embodiment of the invention;

FIG. 24 is a perspective view of an embodiment of the invention;

FIG. 25A is an elevation of an embodiment of the invention;

FIG. 25B is a side elevation of an embodiment of the invention;

FIG. 26A is an elevation of the embodiment of the invention of FIG. 25A in a second configuration;

FIG. 26B is a side elevation of the embodiment of the invention of FIG. 25B in a second configuration;

FIG. 27 is a perspective view of an embodiment of the invention;

FIG. 28 is a perspective view of an embodiment of the invention; and,

FIG. 29 is a perspective view of an embodiment of the invention.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

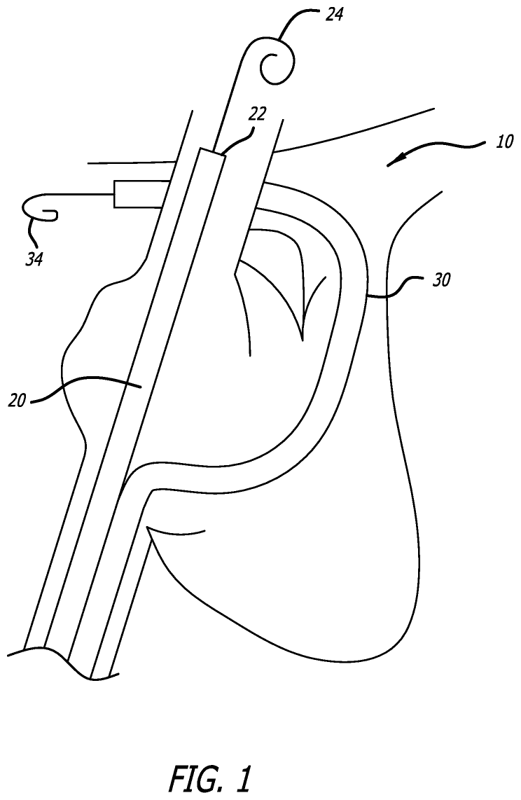

Referring now to the figures and first to FIG. 1, there is shown an example of an application of a magnetic catheter device system 10 of the present invention. Like the other various systems described herein, system 10 generally includes a first catheter 20 and a second catheter 30. Each catheter 20 and 30 include magnetic features 22 and 32, respectively, near their distal ends that allow the catheters to “find” each other, even though traveling in different, but adjacent, lumens. If the lumens are not sufficiently adjacent, further manipulation by the operator may be used to bring them within magnetic attraction proximity of each other.

In the example of FIG. 1, the first catheter 20 is being navigated through the superior vena cava (SVC) and the second catheter 30 is being navigated through the pulmonary artery (PA). Navigation may be assisted by the use of guidewires 24 and 34, respectively. The guidewires 24 and 34 are used first to more easily, and atraumatically, navigate through the tortuous path to, and just beyond, the target location, using known techniques. Care is taken to ensure that the guidewires are extended at least slightly past the target location.

Once the guidewires are in place, the first and second catheters 20 and 30 may be advanced over the guidewires to the target location. Fluoroscopy or other imaging methods may be used to ensure proper locations have been reached. Once established, the magnetic features 22 and 32 may be “activated” or brought into attractive proximity so that they connect across the vascular tissues. Mechanical connection may then be established as described in more detail below.

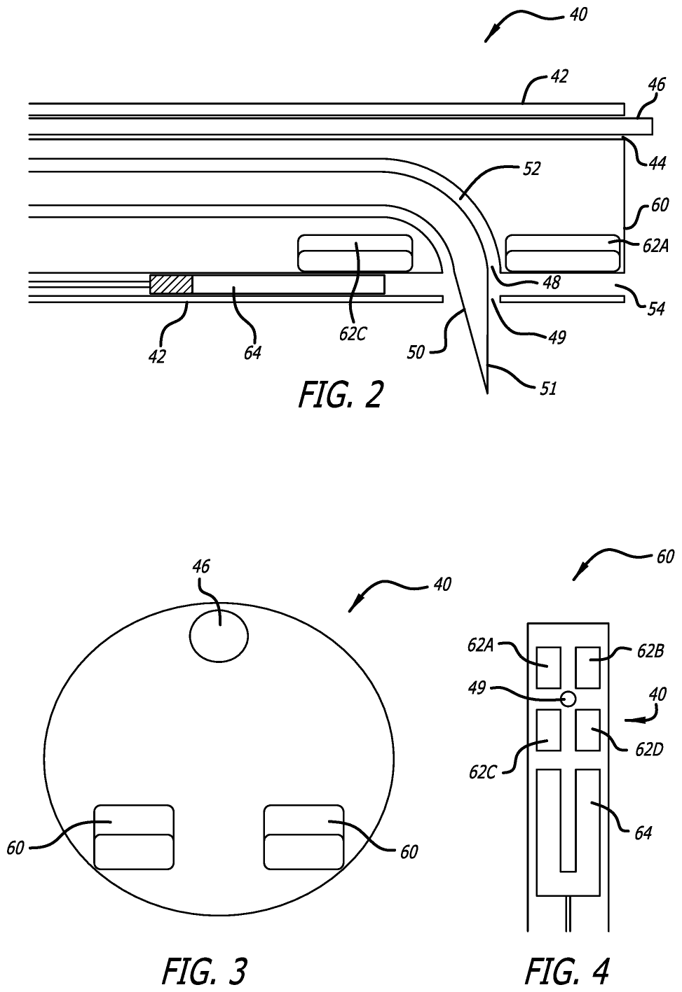

FIGS. 2-11 show an embodiment 40 for use with the aforementioned concept. FIGS. 2 and 3 show a distal end of a first catheter 42. The first catheter 42 generally includes a guidewire lumen 44 useable to follow a guidewire 46, a tool lumen 48, a tool 50, and a magnetic feature 60. The tool lumen 48 may include a turn 52 to direct the tool 50 to exit a side of the catheter 42 in order to access the lumen wall, thus obviating the need to turn the distal end of the catheter toward the lumen wall. This bend 52 also allows the placement of the magnetic feature on a side of the catheter rather than on the distal end, where space may be more limited. This location may also allow a smaller diameter catheter to be used. The tool lumen 48 terminates at a tool aperture 49 through which a tool 50 may emerge when advanced.

The tool 50 may be a needle, wire, probe, RF wire, blade, or other puncture or cutting device. Further more, any or all of the tools herein may include a magnetic tip that would be attracted to the magnet of the receiving side to prevent the tool from hitting the vessel walls The tool 50 is used to puncture tissue and create a passage from the first lumen to the second lumen, and to establish a connection between the two lumens. In the embodiment of FIGS. 2-11, the tool 50 is a needle 51 and a wire 53 advanceable through the needle 51.

The magnetic feature 60 of the first catheter 42 includes at least one, preferably two or more magnets 62. In the embodiment of FIG. 2-11, four magnets 62A, 62B, 62C, and 62D are used. The magnets 62 of the first catheter 42 are arranged such that their polarities are configured to mate with the magnetic feature of the second catheter, discussed below. Thus, in the embodiment shown in FIGS. 2-4, the polarities of the four magnets 62A-D are all arranged similarly, such that the same pole (N for example) for each magnet is facing the same direction. However, one skilled in the art will realize that various arrangements may be used, as long as a complimentary arrangement is used on the second catheter. Thus, if all of the magnets of the first catheter have their N poles facing toward the lumen wall, the magnets of the second catheter will require that their S poles are facing toward the lumen wall.

The magnetic feature 60 of the first catheter 42 may further include a moveable magnetic shield 64. The magnetic shield 64 is advanceable and retractable within a shield lumen 54 of the first catheter 42. The magnetic shield 64 may be a ferromagnetic material or other material that interrupts or blocks the magnetic field of the magnets 62. Thus, advancing the shield to a distal position between the magnetic features of the first and second catheters allows the catheters to be separated without risking tissue damage. In one example, the magnetic shield is a magnet that has its poles arranged to repel the magnetic features or one or both catheters. The magnetic shield 64 may be a set of magnets of similar size, shape and strength magnets 62, or may be slightly weaker while still allowing atraumatic separation. Alternatively, the shield 64 may be formed as an inner or outer sheath that blocks the magnetic field sufficiently to effect atraumatic separation. The magnetic shield and all other magnetic disengagement systems described herein may be operated by a device that provides the operator with a mechanical advantage such as a knob or lever.

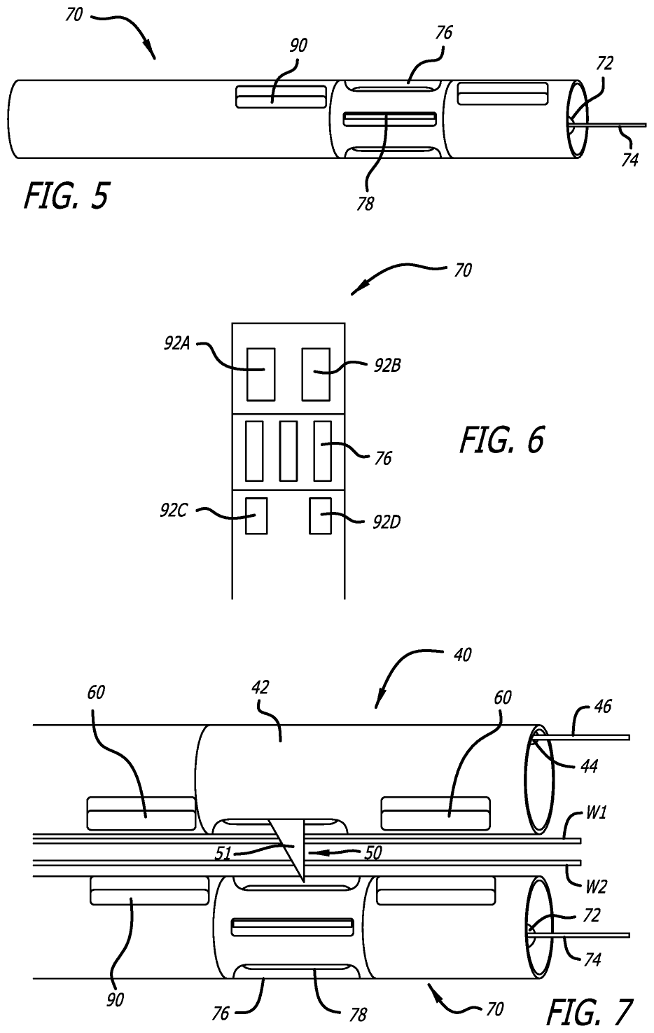

FIGS. 5-6 depicts an embodiment of a second catheter 70. The second catheter 70 is complementarily constructed with regard to the first catheter 42. The second catheter 70 generally includes a guidewire lumen 72 useable to follow a guidewire 74, a tool receiver 76, a tool retaining mechanism 78, and a magnetic feature 90.

The tool receiver 76 is used to allow the tool 50 of the first catheter 42 to enter the second catheter 70. In at least one embodiment, the tool receiver may be a lumen forgivably sized to receive the tool 50. FIG. 5 shows the receiver 76 formed as an aperture in the shape of an elongate slot. The slot 76 is sized to allow slight errors in the alignment of the magnets, without sacrificing structural integrity to the catheter 70. More than one slot may be provided as shown, such that the tool 50 may pass completely through the second catheter 70. Providing a plurality of slots decreases the chances of the tool becoming stuck within the second catheter 70, if such occurrence is undesirable.

The tool retaining mechanism 78 functions to grab the tool once received by the tool receiver 76. The tool retaining mechanism 78 is shown in FIGS. 4-9 is an inner sheath 80 having a plurality of apertures 82 that are sized and shaped to complement the slots 76. The sheath 80 is rotatable within the second catheter 70 such that passageways 84 leading into and out of the second catheter 70 are created when the sheath 80 is rotated such that the slots 76 and apertures 82 are in alignment. When the sheath 80 is rotated, the passageways 84 close. If a tool 50 is located in the passageway 84 while the sheath 80 is rotated, the tool 50 will be pinched between the sheath 80 and the catheter 70 and thus held in place.

The magnetic feature 90 of the second catheter 70 includes at least one, preferably two or more magnets 92. The number of magnets 92 on the second catheter 70 is dictated by the number of magnets 62 on the first catheter 42. In the embodiment of FIG. 2-11, four magnets 62A, 62B, 62C, and 62D are used on the first catheter 42 (not including any magnets that may be used on the shield 64) so four magnets 92A, 92B, 92C and 92D are used on the second catheter 70. The magnets 92 of the second catheter 70 are arranged such that their polarities are configured to mate with the magnetic feature 60 of the first catheter 42. Thus, in the embodiment shown, the polarities of the four magnets 92A-D are all arranged similarly, such that the same pole (S in this example) for each magnet is facing the same direction. However, one skilled in the art will realize that various arrangements may be used, as long as a complimentary arrangement is used on the second catheter. Thus, if all of the magnets of the first catheter have their N poles facing toward the lumen wall, the magnets of the second catheter will require that their S poles are facing toward the lumen wall.

The magnetic feature 90 of the second catheter 70 may optionally include a moveable magnetic shield (not shown) but this would likely unnecessary as the function of interrupting the field would preferably be accomplished by shield 64 of the first catheter 42. Alternatively, the shield feature could be supplied on the second catheter 70 instead of the first catheter 42.

Referring to FIGS. 7-11, the operation of the embodiment 40 can be described in more detail. As seen in FIG. 7, the first catheter 42 and the second catheter 70 have been navigated through respective anatomical lumens or cavities and have been placed in operational proximity across adjacent tissue walls W1 and W2 of the lumens or cavities. Magnetic features 60 and 90 are thus in attractive proximity to each other and have thus aligned the first catheter 42 and the second catheter 70 such that the tool aperture 49 of the first catheter 42 is in alignment with the tool receiver 76 of the second catheter 70.

The needle 51 of the tool 50 has advanced and emerged from the tool aperture 49 and has punctured the walls W1 and W2, as directed by the shape of the tool lumen 48. The needle 51 is aligned with, and has begun to enter, the tool receiver 76 of the second catheter 70.

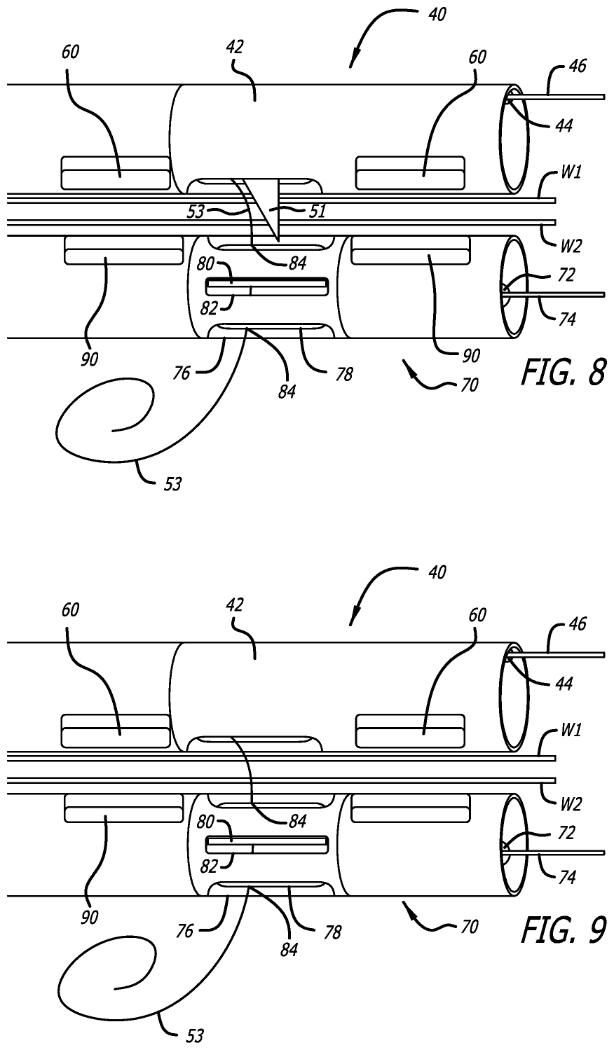

Next, as shown in FIG. 8, the wire 53 of the tool 50 is advanced through the needle 51 and passes through the second catheter 70 via a passageway 84 formed by the positions of the apertures 82 of the inner sheath 80 relative to the slots 76.

In FIG. 9 the needle 51 has been retracted and the wire 53 is left in place, establishing a connection between the first catheter 42 and the second catheter 70, as well as between the first lumen or cavity and the second lumen or cavity.

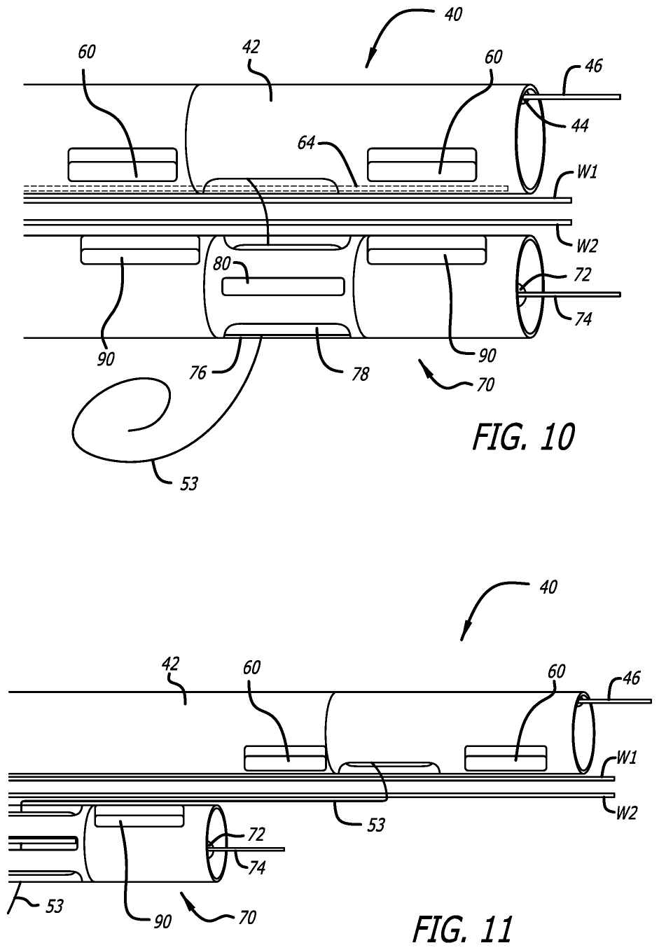

In FIG. 10, the tool retaining mechanism 78 is closed by rotating the inner sheath 80, thereby at least partially closing the passageway 84 and establishing an interference connection between the wire 53 from the first catheter 42 and the tool retaining mechanism of the 78 of the second catheter 70.

Next, the magnetic shield 64 is advanced until located between the magnets 62 of the first catheter 42 and the magnets 92 of the second catheter 70. Having severed the magnetic relationship between the two sets of magnets 62 and 92, the second catheter is retracted, pulling the wire 53 with it, as seen in FIG. 11. The mechanical connection established by the wire 53 may then be used to form a shunt across the walls W1 and W2 percutaneously.

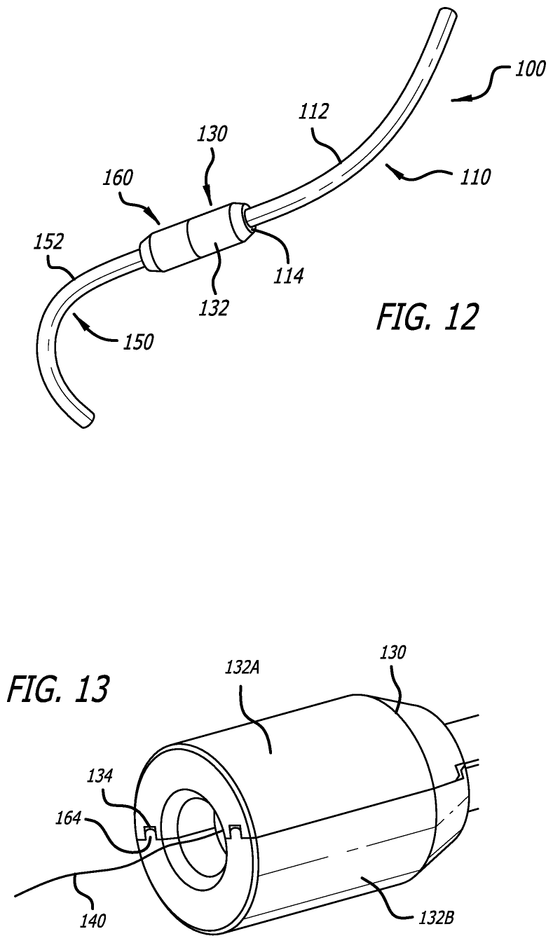

Referring now to FIGS. 12-15, an embodiment 100 of the invention is shown. The embodiment 100 includes a first catheter 110 and a second catheter 150. The first catheter 110 has a body 112 and a magnetic feature 130. The magnetic feature 130 is located at a distal end 114 of the catheter 110 and includes one or more magnets 132. As shown in FIGS. 12-15, the magnetic feature 130 includes two magnets 132A and 132B. Each of the magnets 132A and 132B are cylindrical and each extends halfway around the circumference of the catheter body 112.

The second catheter 150 is similar to the first catheter 110 and includes a body 152 and a second magnetic feature 160 that is constructed and arranged to be attracted to the first magnetic feature 130 in an axial configuration as shown. A tool 140, such as a wire (FIG. 13) is associated with one of the catheters 110 or 150 and is advanced through one catheter into the other across tissues held between the two catheters 110 and 150 once they are magnetically connected across tissue walls, as will be described in greater detail below.

The magnetic features 130 and 160 may be identical with the exception of polar orientation. The halves of each magnetic feature have opposite poles such that they are attracted to each other and remain together as a complete circle or cylinder until forcibly separated. Similarly, the upper (for sake of explanation only) half 132A of the first magnetic feature 130, is a polar opposite of the corresponding upper half 162A of the second magnetic feature 160. Thus, if magnet 132A has a N designation, for example, 132B would be S, 162A would be S and 162B would be N. Additionally. as shown in FIG. 13, there may be a keying feature 134 and 164 on each magnetic feature 130 and 160, respectively, to ensure complete alignment of the two halves. This is shown in FIG. 13 as a tongue and groove joint, but any mating shapes could be used.

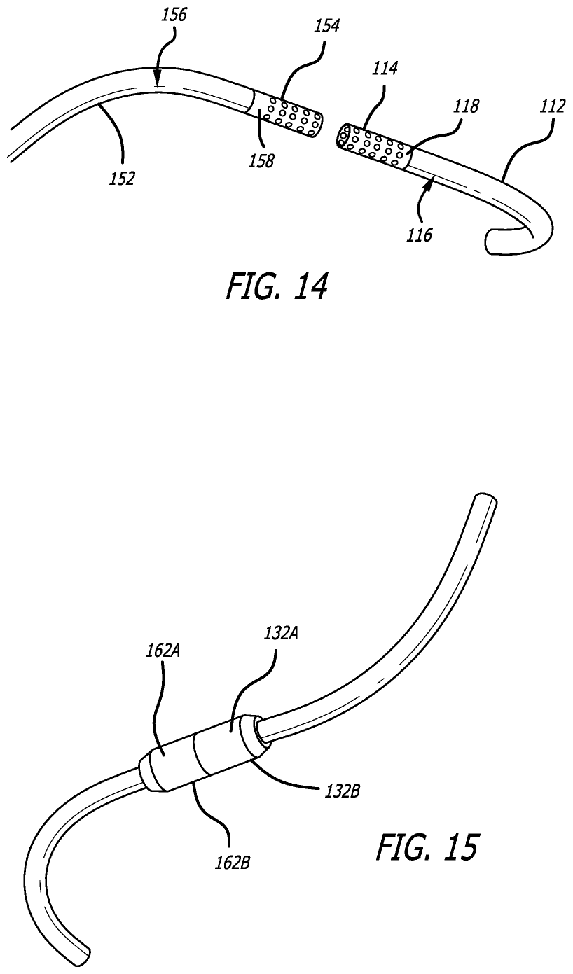

The first and second catheter bodies 112 and 152 have distal ends 114 and 154 that can be seen without the magnetic feature 130 and 160 in FIG. 14. The catheter bodies 112 and 152 have a laser cut spine 116 and 156, respectively, which allows preferential bending or flexing of the catheter in a preferred direction in a manner known in the art. In the embodiment shown in FIG. 14, the spines 116, 156 are long the mid-plane of the catheter thus facilitating preferential bending about that plane The catheter also has distal halves 118 and 158 that are rotated 90 degrees relative to each other to accommodate a 90-degree anatomy. The holes at the ends of the distal halves 118, 158 are useful during manufacturing since the flowing (e.g., melted) material (e.g., Pebax, polyurethane, etc.) that forms a coat/sheath over the distal halves flows/melts into the holes and thereby fixates the coat/sheath to the distal halves.

The design and orientation of the magnetic features 130 and 160 accommodate the placement of a balloon-expanded shunt in the form of a rivet, stent, or other anastomotic—forming implant (not shown). In use, each of the first and second catheters 110 and 150 are navigated to target locations in adjacent bodily spaces, such as a lumen or cavity, that are desired to be joined. The catheters 110 and 150 are either rotated up to 90 degrees toward each other, either via a steering mechanism or a preformed bend that may be released from a straightened configuration, such that the two distal ends 114 and 154 face each other in an axial alignment. This configuration aligns the two central lumens of the catheters and magnetic features, allowing a tool, such as a needle, RF wire, or sharpened guidewire, to be advanced through the first catheter, second catheter, or both, to puncture the tissue walls trapped between the magnetic features 130 and 160. The magnetic features 130 and 160 are strong enough to hold the tissue walls together to form a seal, preventing any of the fluids traveling through the bodily spaced from escaping.

Once the tissue walls are punctured by the tool, an expandable implant can be advanced and centered within the magnets. In at least one embodiment, an interaction between the magnets and the implant sends an electronic or visual signal indicating proper placement of the implant.

Having verified the proper placement of the implant, a balloon may be advanced to the center of the implant, or the balloon may have been advanced with the implant and expanded. Expanding the balloon expands the implant and causes the implant to form a connection between the two body spaces, as well as causing the magnetic features 130 and 160 to spread apart and disengage each other. The permanent connection between the magnetic components 132 and 162 of the magnetic features 130 and 160 and the distal halves 118 and 150 of the distal ends 114 and 154 prevent the magnetic components 132 and 162 from separating from the catheters 110 and 150.

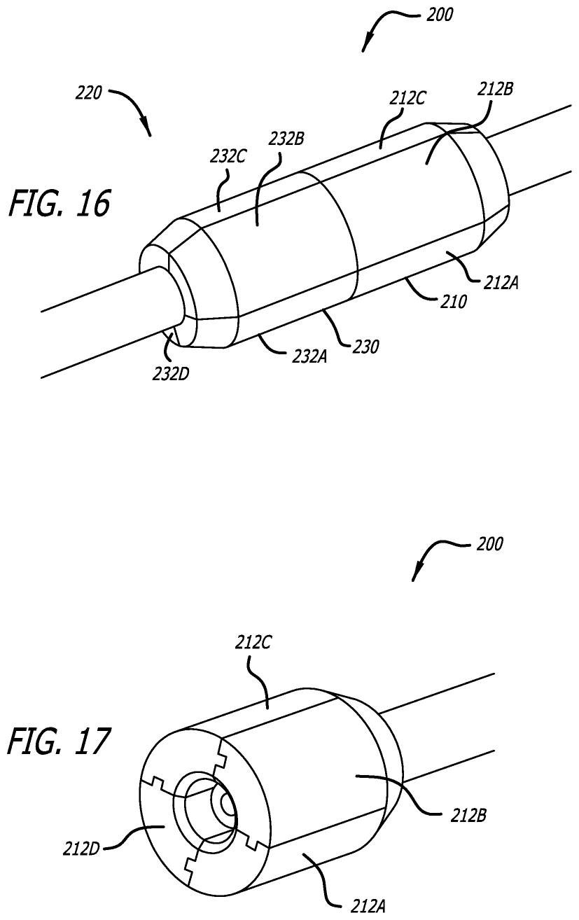

One embodiment 190, shown in FIGS. 16-17, includes first and second catheters 200 and 220 that each include distal cylindrical magnetic features 210 and 230 similar to the embodiments of FIGS. 12-13 except that each use cylindrical quarter magnets 212A-D and 232A-D. These magnets are similarly oriented such that each circumjacent quarter is oppositely polarized, and each axially adjacent quarter is oppositely polarized when joined. The method of use of embodiment 190 is the same as the embodiment 100.

The specific target location may give rise to different embodiments. For example, some adjoining tissue walls are already externally adhered to each other. Take for example, the pulmonary artery (PA) and the superior vena cava (SVC). Because these are externally adhered, the strength of the magnets may be reduced, as they do not have to form a seal, and the risk of injury to the therapy site is reduced during separation. In this case the purpose of the magnetic features is simply to locate where the vessels cross, and to protect the receiving vessel during puncture. Thus, the cylindrical magnets on each catheter may be a complete cylinder, without sections, and the procedure may be to puncture across the tissue with a wire, remove the magnet catheters, track a balloon catheter over the wire, and expand the balloon and implant. Similarly, it the target locations involve tissues that self-fuse when magnetic compression is applied, the need for covered implants and laser cut breakaway designs may be obviated.

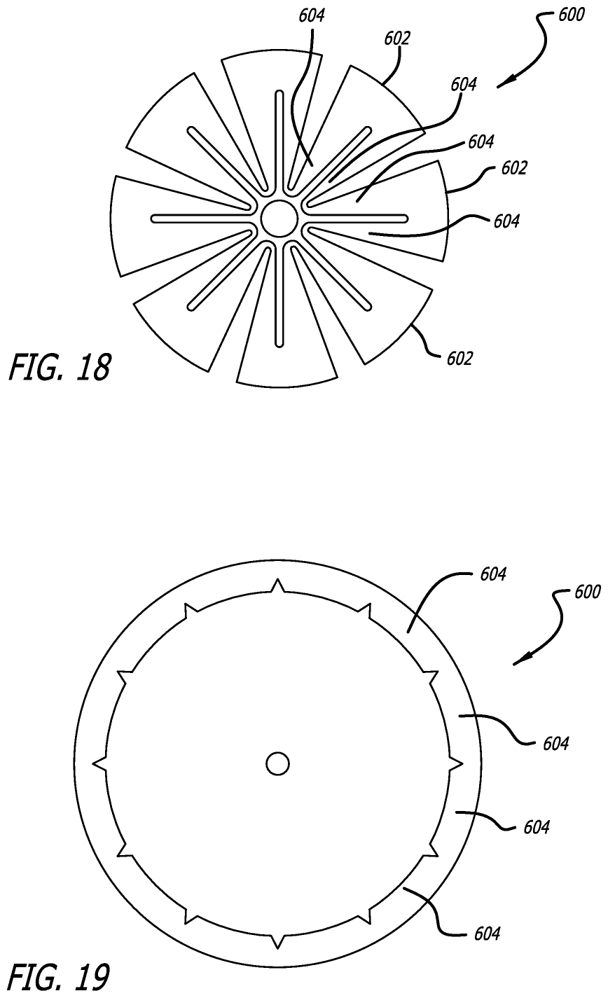

FIGS. 18-19 show a low-profile magnetic feature 600 that may be used with any of the embodiments discussed herein, and especially with the end-to-end embodiments. The magnetic feature 600 may be folded into the configuration shown in the figure, creating a star-shape with radiating folding sections 602. In at least one embodiment, each of the individual components 604 are in a polar alignment that causes a repellant effect in the folded configuration. Thus, upon release from a catheter, the magnetic feature 600 automatically expands into the circular configuration shown in FIG. 19. Alternatively, the covering used to contain the magnets may have a resilient property that self-expands regardless of any resistance created by the magnets.

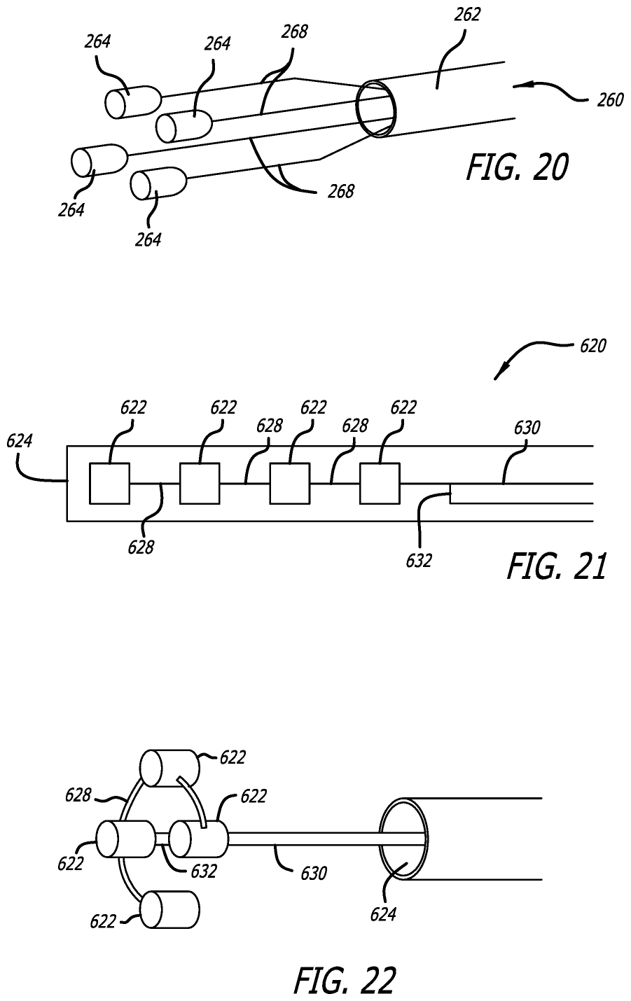

FIG. 20 shows an embodiment 260 of a catheter 262 that houses a plurality of large magnets 264 that are nearly the size of the catheter lumen 266. FIG. 20 demonstrates that magnetic force may be increased for a given delivery catheter size if the magnets are tethered, such as by tethers 268 and then arranged axially within the catheter 262.

FIGS. 21-22 show another low-profile embodiment of a magnetic feature 620 that may be used with any of the embodiments discussed herein. This magnetic feature 620 includes a plurality of magnets 622 that a sized slightly smaller than a lumen 624 of a delivery catheter 626. The magnets 622 are connected via a shape set connecting ring 628 that allows axial alignment of the magnets 622 when loaded into the catheter 626, but assumes a ring shaped configuration, as seen in FIG. 22, when deployed. An inner catheter 630 with a guidewire lumen 632 doubles as a pusher catheter to deploy the magnets 622.

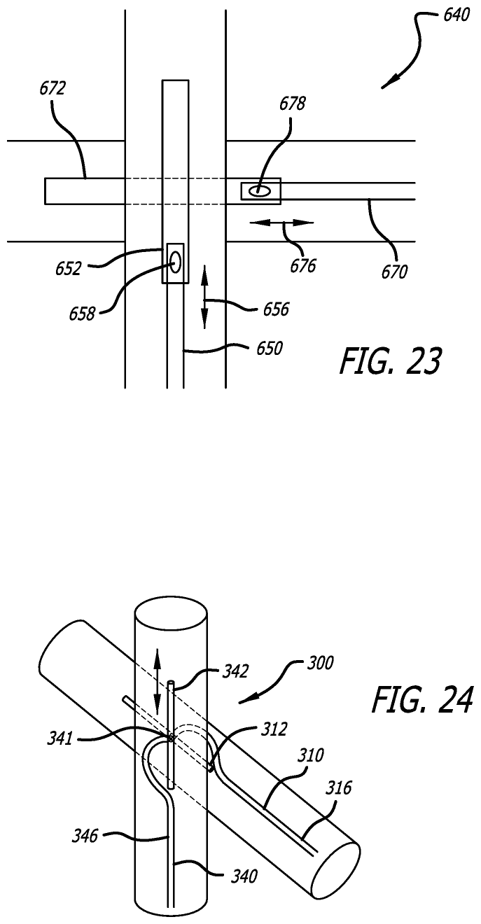

FIG. 23 shows a “cross” embodiment 640 of a system of the invention. This cross embodiment 640 includes a first catheter 650 and a second embodiment 670. A magnetic feature 652 and 672 are associated with each catheter and include an elongate magnet 654 and 674 each with a central lumen or telescoping feature 656 and 676, and a side port 658 and 678. The elongate magnets 652 and 672 are advanced through lumens such as those of the SVC and PA, and are attracted to each other when they cross. They are then used to guide the advancement of the associated catheters 650 and 670 until the side ports 658 and 678 align and can be used to establish a mechanical connection using a needle or sharpened/RF guidewire as discussed above.

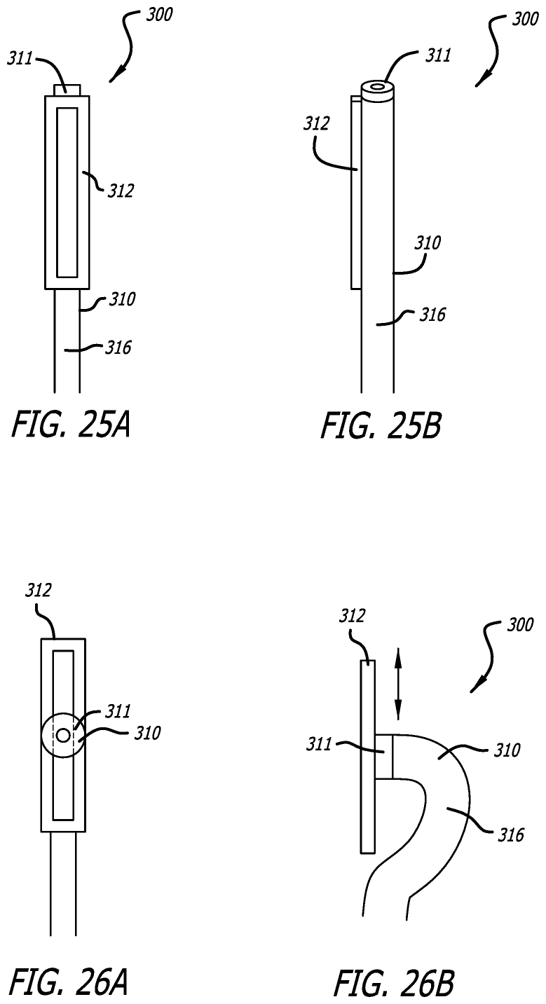

FIGS. 24-26 show an alternative “cross” embodiment 300 in which the distal ends of catheters 310 and 340 have magnets 311 and 341 that are slidingly associated with elongate magnets 312 and 342 such that when the catheters 310 and 340 retracted relative to the magnets 312 and 342, the distal ends 314 and 344 rotate toward the magnets, and thus into an axial alignment with each other. The lumens 316 and 346 of the catheters 310 and 340 thus align to form a passage way through which a mechanical connection can be established as described above. FIGS. 25 and 26 are close up views of the first catheter 310 but first and second catheters 310 and 340 are identical other than magnet polarity orientation.

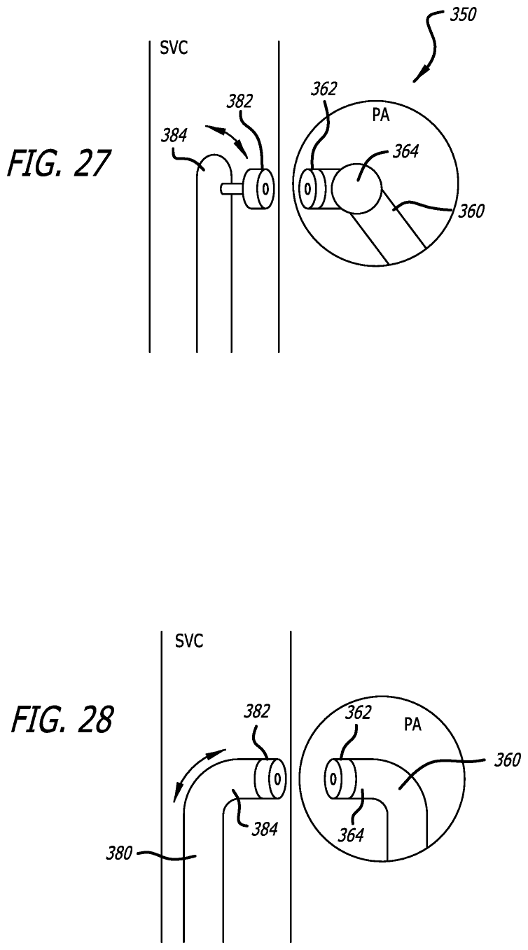

FIGS. 27 and 28 show an embodiment 350 of a device that includes first and second catheters 360 and 380 that have hinged magnetic features 362 and 382 on their distal ends. The magnetic features 362 and 382 remain in axial alignment with the catheters 360 and 380 until they come into attractive proximity to each other, at which time they hinge or rotate around the ends 364 and 384 of the catheters 360 and 380 to face each other. Once connected, the catheters can be flexed to align central lumens 366 and 386 of the catheters 360 and 380 such that a tool can be advanced therethrough to form a mechanical connection as described above.

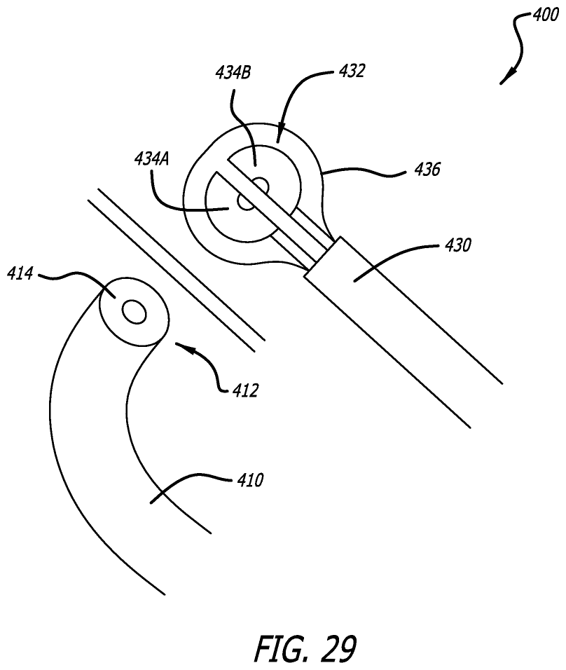

FIG. 29 shows another embodiment 400 that includes a first catheter 410 having a distal magnetic feature 412 in the form of an axially aligned cylindrical magnet 414 having a lumen 418. The second catheter 430 includes magnetic features 432 in the form of a pair of split magnets 434A and 434B that may be similarly aligned or aligned at angle, up to 90 degrees, to the catheter body 430. The magnets 434A and B may spread apart from each other in order to accept a tool, such as a wire, that extends from the first catheter 410. The second catheter 430 is further equipped with a snare 436 that surrounds the magnetic feature 432 and can be tightened to capture with wire, thereby establishing a mechanical connection.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A system for establishing a connection across tissue walls of adjacent anatomical spaces comprising:
a first catheter having a first lumen and carrying a tool in said lumen;
a second catheter having a second lumen;
a first magnetic feature on a distal end of said first catheter;
a second magnetic feature on a distal end of said second catheter;
wherein said first magnetic feature includes a polar arrangement configured to attract said second magnetic feature;
a first aperture on said first catheter and a second aperture on said second catheter;
wherein said first and second apertures align to form a passageway when said first and second magnetic features are connected across said tissue walls;
wherein said tool is positioned to puncture said tissue walls when advanced through said first lumen;
a tool receiver associated with said second catheter for engaging the tool after it crosses through said tissue walls in order to establish a connection across the tissue walls.

2. The system of claim 1 wherein said tool comprises a needle.

3. The system of claim 1 wherein the tool comprises a wire.

4. The system of claim 1 wherein the tool comprises an RF wire.

5. The system of claim 1 further comprising a tool retaining mechanism.

6. The system of claim 5 wherein said tool retaining mechanism comprises a component rotatable relative to said second catheter.

7. The system of claim 5 wherein said tool retaining mechanism comprises a snare.

8. The system of claim 1 further comprising a magnetic disengagement mechanism.

9. The system of claim 8 wherein said magnetic disengagement mechanism comprises a slidable magnet shield.

10. The system of claim 8 wherein said magnetic disengagement mechanism comprises a balloon.

11. A method of forming a connection across tissue walls of adjacent anatomical spaces comprising:
navigating a first catheter to a first target location in a first anatomical space;
navigating a second catheter to a second target location in second anatomical space adjacent said first anatomical space;
using magnetic force to create a connection between said first and second catheter across tissue walls of said first and second anatomical spaces;
passing a tool through the tissue walls from said first catheter to said second catheter thereby establishing a connection;
removing said first and second catheters.

12. The method of claim 11 wherein removing said first and second catheters comprises interrupting said magnetic force, thereby allowing atraumatic removal of said first and second catheters.

13. The method of claim 11 wherein passing a tool through the tissue walls comprises passing a wire through said tissue walls.

14. The method of claim 11 wherein passing a tool through the tissue walls comprises using a needle to puncture said tissue walls and passing a wire through a lumen of said needle.

15. The method of claim 12 wherein interrupting said magnetic force comprises advancing a shield between magnetic features of said first and second catheters, thereby blocking said magnetic force.

16. The method of claim 12 wherein interrupting said magnetic force comprises inflating a balloon within magnets providing said magnetic force, thus separating the magnets from each other.

17. The method of claim 11 wherein establishing a connection further comprises grabbing said tool using a tool retaining mechanism.

18. A system for establishing a connection across tissue walls of adjacent anatomical spaces comprising:
a first navigable device having a first magnetic feature on a distal end thereof;
a second navigable device having a second magnetic feature on a distal end thereof;
wherein said first and second magnetic features are attracted to each other across tissue walls such that said distal ends of said first and second navigable devices become temporarily fixed to each other when brought into magnetic proximity of each other;
a tool passable through tissue from one of said first and second navigable devices to the other, thereby establishing a connection across the tissue walls.

19. The system of claim 18 wherein said first and second magnetic features form a passageway through said first and second features when properly aligned, through which said tool may pass.

20. The system of claim 18 further comprising a magnetic disengagement feature.
